(19) United States
(12) Patent Application Publication
(10) Pub. No.: US 2008/0132843 A1
(43) Pub. Date: Jun. 5, 2008

(54) Syringe assembly and an infusion pump assembly incorporating such

(75) Inventor: Bahram Sharifi, Schwenksville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Animas Corporation

(21) Appl. No.: 11/633,160

(22) Filed: Dec. 4, 2006

(51) Int. Cl. A61M 5/142

(52) U.S. Cl. 604/151

(57) ABSTRACT

A syringe assembly for use with an infusion pump having a drive piston. The syringe assembly comprises a substantially hollow syringe housing and a plunger axially movable within the syringe housing to expel a fluid therefrom. The plunger is configured to operatively engage the drive piston via relative axial movement. The plunger has a body with a radially elastic sealing member extending therefrom and configured to engage a portion of the drive piston aligned therewith to seal the plunger relative to the drive piston.

### FIELD OF THE INVENTION

The present invention relates to an infusion pump assembly for controlled delivery of a pharmaceutical product to a subject, and more specifically to a syringe assembly for use with the infusion pump.

### BACKGROUND OF THE INVENTION

Infusion pumps provide a significant lifestyle benefit for individuals requiring multiple deliveries of volumetrically proportioned medication to their body over a period of time. Infusion pumps reliably dispense the required medication to the patient through an infusion path established between the patient and the pump. The infusion path is a conduit secured to the pump at one end and secured intravenously or subcutaneously to a patient on the other. The operation of the infusion pump is controlled by a processor. The processor controls the delivery of periodic dosages of medication to a patient at predetermined times. Thus, a patient is able to rely on the infusion pump for delivering the required dosage of medication intravenously or subcutaneously over a period of time. In this way, the patient need not interrupt life activities for repeated manual delivery of required medication.

As is known, infusion pumps often employ a piston-type drive mechanism for urging the contents of a pharmaceutical cartridge or “syringe assembly” internal to the pump along the infusion path to the subject. The piston-type drive selectively drives the syringe plunger to dispense a desired amount of fluid from the syringe housing.

### SUMMARY OF THE INVENTION

The features and advantages of the invention will be set forth in the description which follows, and in part will be apparent from the description, or may be learned by practice of the invention. The advantages of the invention will be realized and attained by the apparatus, and the method of practicing the invention, particularly pointed out in the written description and claims below, as well as in the attached drawings.

In accordance with an aspect of the invention, a syringe assembly for use with an infusion pump having a drive piston is provided. The syringe assembly comprises a substantially hollow syringe housing and a plunger axially movable within the syringe housing to expel a fluid therefrom. The plunger has a body with a radially elastic sealing member extending therefrom and configured to engage a portion of the drive piston aligned therewith to seal the plunger relative to the drive piston.

In accordance with another aspect of the invention, an infusion pump assembly is provided. The infusion pump assembly comprises an insulin pump including a drive piston and a syringe assembly including a substantially hollow syringe housing and a plunger axially movable within the syringe housing to expel a fluid therefrom. The plunger has a body with a portion configured to engage the drive piston. A sealing elastic member is configured to be positioned between the plunger body and the drive piston to seal the plunger body relative to the drive piston.

In accordance with another aspect of the invention, the plunger is configured to receive the drive piston with an annular gap between the drive body and the plunger hollow body and the sealing elastic member is positional within the gap to seal the plunger relative to the drive piston.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there is shown various embodiments. It should be understood, however, that the invention is not limited to the precise arrangements and instrumentalities shown.

In the drawings:

FIG. 1 is side elevation view of an exemplary infusion pump incorporating an embodiment of the present invention, the infusion pump having a wall of its casing removed to show the layout of the components therein;

FIG. 2 is a side elevation view of the infusion pump shown in FIG. 1 with the wall in place and the pump door open;

FIG. 3 is an end elevation view of the infusion pump with the pump door open for loading a syringe assembly;

FIG. 4 is an isometric view of a syringe assembly in accordance with an exemplary embodiment of the present invention;

FIG. 5 is a an exploded cross-sectional view of the syringe assembly of FIG. 4 illustrating the sealing insert prior to assembly;

FIG. 6 is a cross-sectional view along the line 6-6 in FIG. 4;

FIG. 7 is a cross-sectional view similar to FIG. 6 of a syringe assembly in accordance with an alternative exemplary embodiment of the present invention;

FIG. 8 is a cross-sectional view of a drive piston in accordance with an exemplary embodiment of the invention;

FIG. 9 is a side elevation view in partial section illustrating insertion of the syringe assembly into the infusion pump casing;

FIG. 10 is a cross-sectional view illustrating the syringe assembly of FIG. 9 loaded in an operational position within the infusion pump;

FIG. 11 is a cross-sectional view of a drive piston in accordance with an alternative exemplary embodiment of the invention;

FIG. 12 is a cross-sectional view of a drive piston in accordance with another alternative exemplary embodiment of the invention; and

FIG. 13 is a cross-sectional view similar to FIG. 6 of a syringe assembly in accordance with another alternative exemplary embodiment of the present invention.

### DETAILED DESCRIPTION OF THE INVENTION

Certain terminology is used in the following description for convenience only and is not limiting. The words “right,” “left,” “lower” and “upper” designate directions in the drawings to which reference is made. The words “inwardly” and “outwardly” refer to directions toward and away from, respectively, the geometric center of the infusion pump and designated parts thereof. The terminology includes the words specifically mentioned above, derivatives thereof and words of similar import.

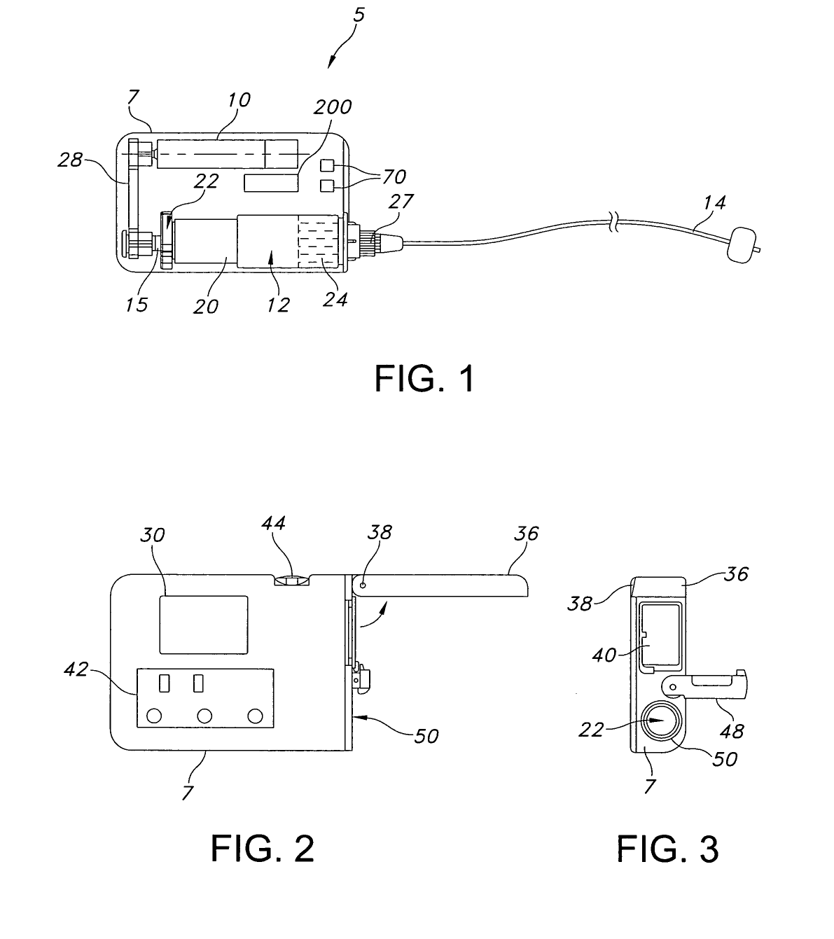

Referring to FIG. 1, piston-type infusion pump 5 in accordance with an exemplary embodiment of the present invention is shown for delivering medication 24 or other fluid (in phantom) to a patient along infusion path 14. Infusion pump 5 desirably includes sealed pump casing 7, processing circuitry 200, power cells 70, motor 10, gear train 28, lead screw 15, and drive piston 22. Syringe assembly 12 is positional within pump casing 7 such that drive piston 22 engages plunger 20 of syringe assembly 12.

In operation, processing circuitry 200, powered by power cells 70, controls the operation of infusion pump 5. Motor 10 is incrementally engaged to infuse medication to a patient at predetermined intervals. Upon engagement, motor 10 causes lead screw 15 to rotate by means of gear train 28. When lead screw 15 is driven by motor 10, drive piston 22 is driven axially toward syringe assembly 12, thereby pushing plunger 20. This causes delivery of medication 24 from within syringe housing 26 of syringe assembly 12. Infusion path 14 is connected by connector 27 to dispensing tip 25 (see FIG. 5) of syringe housing 26 to provide fluidic communication between infusion pump 5 and a patient.

Referring now to FIGS. 2-3, casing 7 of infusion pump 5 is shown. Pump casing 7 is desirably formed of a thermoplastic material and preferably made watertight by sealing any openings therein. Desirably, pump casing 7 supports LCD display 30, keypad 42, priming button 44, battery door 40, hinge 38, pump door latch 48, and pump door 36. To load a syringe assembly 12 within casing 7 pump door 36 is opened to expose the interior infusion port 50 and battery door 40. Syringe assembly 12 is configured to be moved axially through interior infusion port 50 into the interior of pump casing 7 such that plunger 20 axially engages drive piston 22, as will be described in more detail hereinafter. In FIGS. 2 and 3, pump door latch 48 has been rotated away from pump casing 7 in order to release pump door 36 so it may pivot open at hinge 38. Battery door 40 is removable for replacing the power cells 70.

The present invention is not limited to the illustrated infusion pump. Infusion pump 5 may have various configurations including various controls, power sources, drive means, access ports and doors, sizes and shapes.

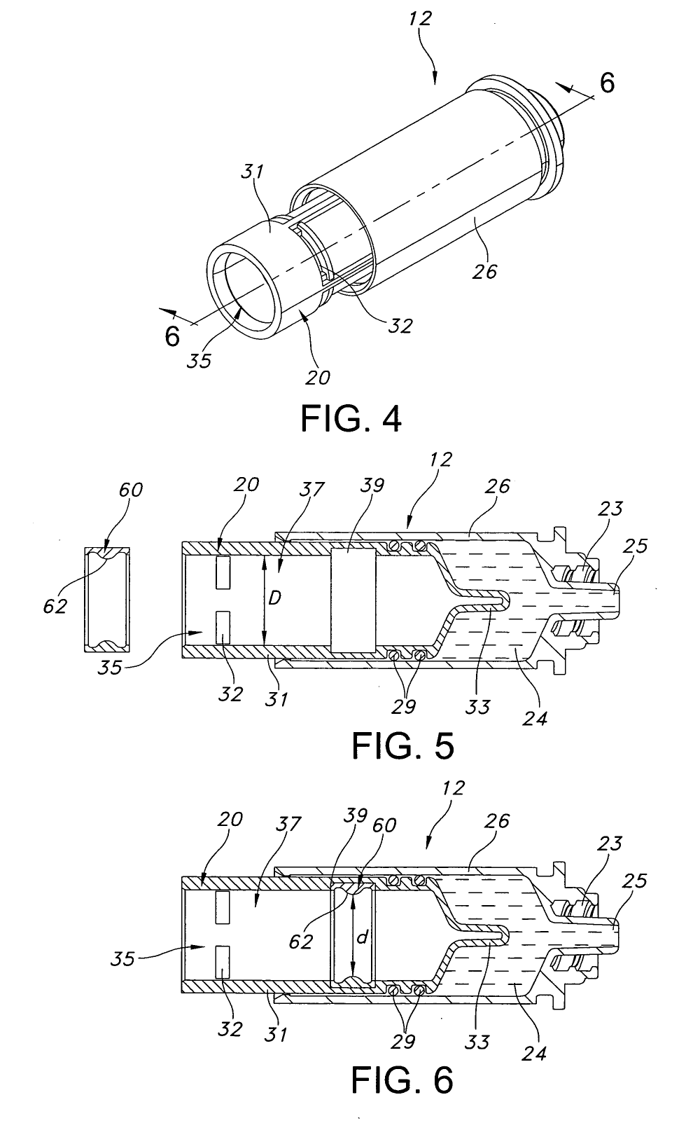

Referring to FIGS. 4-6, syringe assembly 12 includes plunger 20 and syringe housing 26. Seal rings 29 or the like may be provided between plunger 20 and syringe housing 26 to seal medication 24 within syringe housing 26. Upon inward axial motion of plunger 20 relative to syringe housing 26, medication 24 is dispensed through dispensing tip 25. Connection interface 23 is provided adjacent dispensing tip 25 to facilitate connection to connector 27. Various connection interfaces 23 may be provided.

In the present embodiment, plunger 20 includes tubular body 31 which has a generally hollow interior 37 extending between closed end 33 and generally open end 35. Generally open end 35 is configured to receive drive piston 22 as will be described hereinafter. Plunger body 31 may include vent holes 32 or the like to allow pressure to escape as drive piston 22 is advanced into hollow interior 37. Interior annular groove 39 is provided along the interior surface of plunger body 31 and is configured to receive sealing insert 60 including elastic sealing member 62. With sealing insert 60 positioned in annular groove 39, elastic sealing member 62 extends radially inward into hollow interior 37 and has an internal diameter d which is less than the interior diameter D of tubular body 31. Interior annular groove 39 is desirably provided proximate generally open end 35, but may otherwise be positioned. Syringe assembly 12 as illustrated in FIGS. 4 and 6 is pre-filled with medication 24 or the like and is ready for use in infusion pump 5.

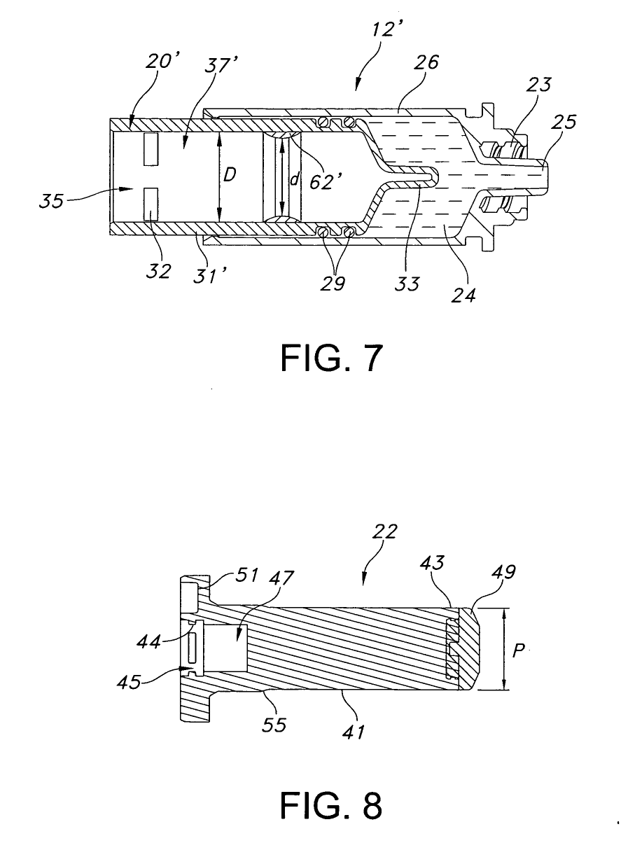

Referring to FIG. 7, syringe assembly 12′ in accordance with an alternative exemplary embodiment of the present invention is shown. Syringe assembly 12′ is substantially the same as in the previous embodiment, however, elastic sealing member 62′ is formed integrally, for example, via overmolding or the like, within hollow interior 37′ of plunger tubular body 31′. As such, an annular groove or the like is not required. In other respects, plunger 20′ is substantially the same as in the previous embodiment. Elastic sealing member 62, 62′ may be provided within plunger 20, 20′ in various other manners other than those described herein

Referring to FIGS. 8-10, drive piston 22 includes tubular body 41 having a hollow interior portion 47 which is configured to receive lead screw 15. Threads 44 adjacent open end 45 of piston body 41 are configured to engage threads 16 of lead screw 15 to facilitate driving motion of drive piston 22. Drive piston 22 is supported within casing 7, either by lead screw 15 alone or via other support structures (not shown). Shoulder 51 may be provided about open end 45 to further support drive piston 22 within casing 7. End 43 of piston body 41 is configured to receive cap 49 which is desirably manufactured from an elastomeric material. Alternatively, end 43 may be manufactured as a closed end or cap 49 may be formed integrally therewith, for example, via overmolding. Between ends 43 and 45, piston body 41 may expand radially outwardly to define shoulder 55 which is configured to engage open end 35 of plunger 20 upon insertion therein. Forward of shoulder 55, piston body 41 has an outer diameter P which is larger than interior diameter d of elastic sealing member 62.

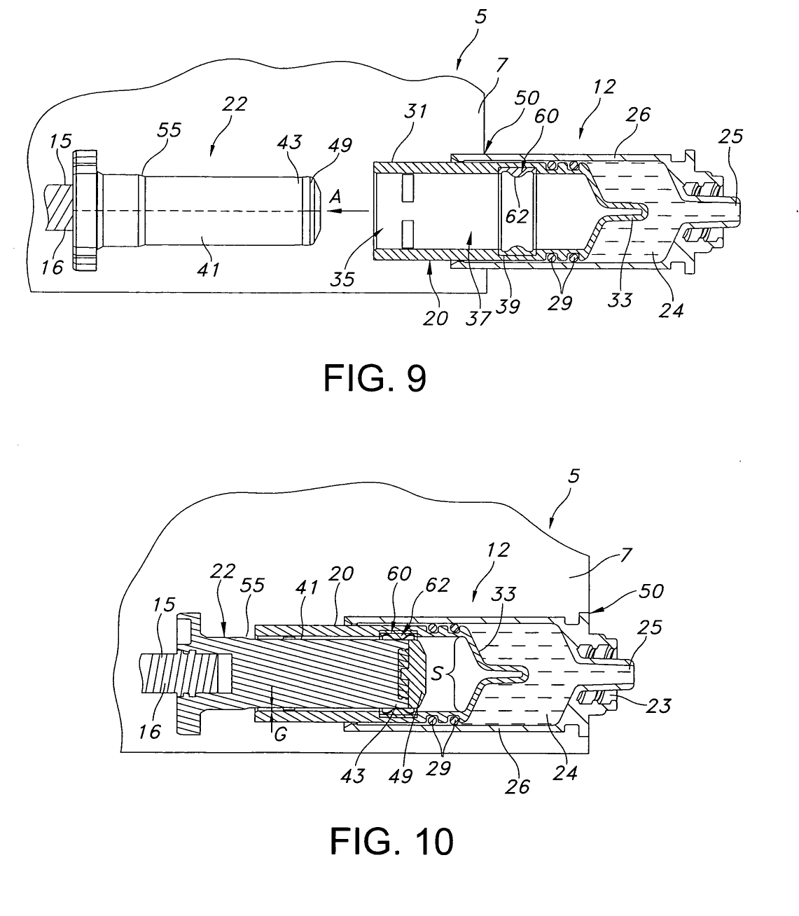

As illustrated in FIG. 9, to install syringe assembly 12 within infusion pump 5, syringe assembly 12 is moved axially through interior infusion port 50, as indicated by arrow A. End 43 of drive piston 22 enters plunger 20 through open end 35 and passes through hollow interior 37 until shoulder 55 engages open end 35. Alternatively, plunger 20 and drive piston 22 may be configured such that drive piston 22 is inserted until cap 49 engages plunger closed end 33. Upon such complete insertion, sealing elastic member 62 engages and provides a seal against drive piston body 41 since piston body 41 has an outer diameter P, at the point of contact with sealing elastic member 62, which is larger than interior diameter d of elastic sealing member 62. Forward of the point of contact, piston body 41 is desirably narrower than interior diameter d of elastic sealing member 62 such that piston body 41 is inserted freely and air is not trapped between drive piston 22 and plunger 20.

The configuration and material of elastic sealing member 62 and the width of gap G are selected such that elastic sealing member 62 creates a friction force between plunger 20 and drive piston 22. The friction force is desirably designed to be greater than a pressure differential or the like which may act on the plunger 20, while still allowing easy separation of the components. In this regard, provision of elastic sealing member 62 reduces the potential pressure differential and thereby reduces the necessary friction force. For example, without elastic sealing member 62, a pressure differential may pass through gap G between plunger body 31 and drive piston body 41 and act on the complete surface S defined at closed end 33 of plunger 20. The force on plunger 20 relative to drive piston 22 which is fixed to lead screw 15 is equal to the pressure differential multiplied by the area upon which the pressure differential acts. The area of closed end 33 can be approximated as A_{max plungerID}=ΠD²/4 wherein D is the inside diameter of plunger 20. In comparison, in the present invention, a pressure differential can only act on the sealing elastic member 62 which has a radial width only equal to the width of gap G. As such, the pressure differential only acts on an area of A_{sealingmember}=Π(D²−P²)/4 wherein P is the outside diameter of drive piston 22. Therefore, the ratio of the A_{sealingmember} to that of the A_{maxplunderID} is equal to

$$\frac{{A}_{sealingmember}}{{A}_{max plungerID}} = ( {D}^{2} - {P}^{2} ) / {D}^{2} .$$

### It is noted that a pressure differential will act on the rear end face of plunger 20, however, since this force will be equal in both scenarios and thereby cancel out, it has been removed from the equations.

In an illustrative example, if plunger inside diameter is 1 inch and drive piston outside diameter P is 90 percent thereof, or 0.9 inches, the ratio of the A_{sealingmember} to that of the A_{maxplunderID} is equal to

$$\frac{{A}_{sealingmember}}{{A}_{max plungerID}} = ( {1}^{2} - {0.9}^{2} ) / {1}^{2}$$

which equals 0.19 or approximately one-fifth the area upon which the pressure differential can act. As a result, with a 10 percent clearance between drive piston 22 and plunger 20, sealing elastic member 62 reduces the resultant force created by the pressure differential by approximately 80 percent. Further reducing the clearance between drive piston 22 and plunger 20 will further reduce the resultant force created by the pressure differential. By lowering the resultant force, for example, by approximately 80 percent, the necessary friction force can also be reduced.

When syringe assembly 12 is to be removed, an axial force opposite arrow A is applied to syringe assembly 12. The axial force will be greater than the friction force created by elastic sealing member 62, however, as described above, the required axial force will be reduced in view of the reduced friction force. The configuration and material of elastic sealing member 62 and the width of gap G are selected to facilitate relatively easy axial insertion and removal of syringe assembly 12 while providing a sufficient friction force and minimizing the effect of a pressure differential force or the like.

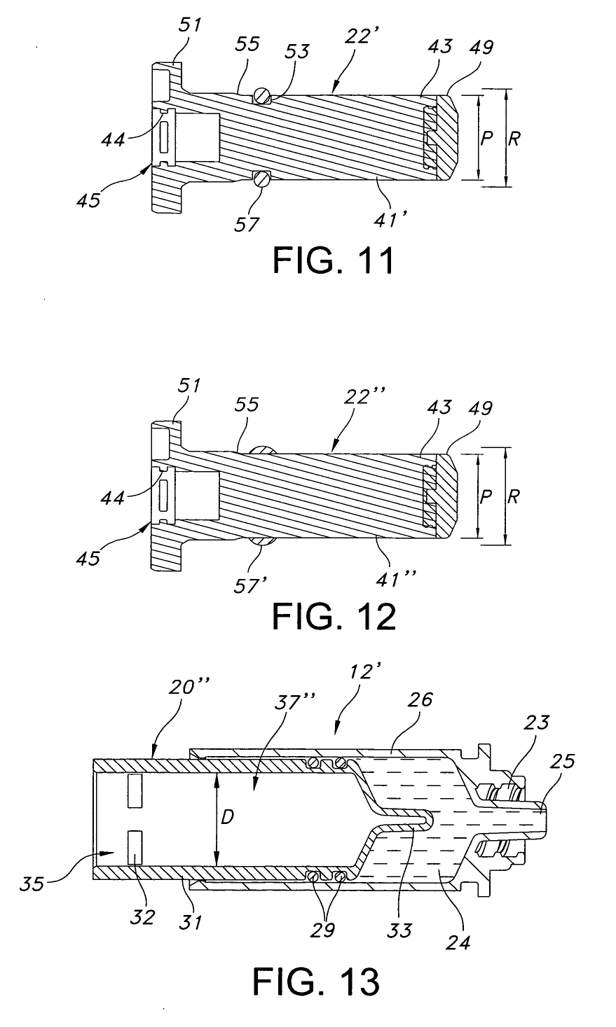

Referring to FIGS. 11-13, alternative exemplary embodiments of the present invention are shown. The present embodiments are similar to the previous embodiments, except that elastic sealing member 57, 57′ is provided on drive piston 22′, 22″ instead of on plunger 20″ of syringe assembly 12″. In each of these embodiments, plunger body 31 may be provided with hollow interior 37″ which has a consistent diameter D without any inwardly extending portions.

With respect to drive piston 22′ shown in FIG. 11, piston body 41′ is provided with exterior annular groove 53 which is configured to receive sealing elastic element 57. Annular groove 53 is desirably provided proximate to shoulder 55. Sealing elastic member 57 and annular groove 53 are configured such that sealing elastic member 57 extends radially outward and has an outer diameter R which is larger than piston body diameter P and plunger inside diameter D. Upon insertion of drive piston 22′, elastic sealing member 57 engages and seals against the inside surface of plunger hollow interior 37″ and provides a reduced area for which a pressure differential or the like to act upon in a similar manner as described above with respect to the first two exemplary embodiments. In general aspects, the infusion pump assembly generally operates as in the previous embodiments.

In the exemplary embodiment illustrated in FIG. 12, sealing elastic member 57′ is formed integrally with body 41″ of drive piston 22″, for example, via overmolding or the like. As in the previous embodiment, sealing elastic member 57′ has an outside diameter R which is larger than piston body diameter P and plunger inside diameter D. In other respects, drive piston 22″ operates in the same manner as described with respect to the above embodiments.

While the various embodiments have been illustrated and described with drive piston 22 extending into plunger 20, the components may be reversed with a portion of the plunger extending into a portion of the drive piston.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A syringe assembly for use with an infusion pump having a drive piston, the syringe assembly comprising:
a substantially hollow syringe housing; and
a plunger axially movable within the syringe housing to expel a fluid therefrom, the plunger being configured to operatively engage the drive piston via relative axial movement, the plunger having a body with a radially elastic sealing member extending therefrom and configured to engage a portion of the drive piston aligned therewith to seal the plunger relative to the drive piston.

2. The syringe assembly of claim 1 wherein the plunger body has a substantially hollow tubular configuration with an open end configured to receive a portion of the drive piston and the sealing elastic member extends radially inward.

3. The syringe assembly of claim 1 wherein the sealing elastic member is provided on an insert configured to be received and retained in an annular groove on the plunger body.

4. The syringe assembly of claim 1 wherein the sealing elastic member is formed integrally with the plunger body.

5. The syringe assembly of claim 1 wherein at least a portion of the plunger body is configured to be received within a portion of the drive piston and the sealing elastic member extends radially outward.

6. An infusion pump assembly comprising:
an insulin pump including a drive piston;
a syringe assembly including a substantially hollow syringe housing and a plunger axially movable within the syringe housing to expel a fluid therefrom, the plunger having a body with a portion configured to operatively engage the drive piston via relative axial movement; and
a sealing elastic member configured to be positioned between the plunger body and the drive piston to seal the plunger body relative to the drive piston.

7. The infusion pump assembly of claim 6 wherein the sealing elastic member is carried on the plunger body.

8. The infusion pump assembly of claim 7 wherein the plunger body includes an annular groove configured to receive and retain an insert which defines the sealing elastic member.

9. The infusion pump assembly of claim 7 wherein the sealing elastic member is formed integrally with the plunger body.

10. The infusion pump assembly of claim 7 wherein the sealing elastic member is positioned on the plunger body such that the sealing elastic member is axially aligned with a forward portion of the drive piston when the plunger is operationally positioned relative to drive piston.

11. The infusion pump assembly of claim 7 wherein the plunger body has a substantially hollow tubular configuration with an open end configured to receive a portion of the drive piston and the sealing elastic member extends from the plunger body radially inwardly.

12. The infusion pump assembly of claim 7 wherein at least a portion of the plunger body is configured to be received within a portion of the drive piston and the sealing elastic member extends from the plunger body radially outwardly.

13. The infusion pump assembly of claim 7 wherein the sealing elastic member is carried on the drive piston.

14. The infusion pump assembly of claim 13 wherein the drive piston includes an annular groove configured to receive and retain the sealing elastic member.

15. The infusion pump assembly of claim 13 wherein the sealing elastic member is formed integrally with the drive piston.

16. The infusion pump assembly of claim 13 wherein the drive piston includes a radial shoulder configured to engage an open end of the plunger when the plunger is operationally positioned relative to drive piston and the sealing elastic member is axially positioned proximate the shoulder.

17. The infusion pump assembly of claim 13 wherein the plunger body has a substantially hollow tubular configuration with an open end configured to receive a portion of the drive piston and the sealing elastic member extends from the drive piston radially outwardly.

18. The infusion pump assembly of claim 13 wherein at least a portion of the plunger body is configured to be received within a portion of the drive piston and the sealing elastic member extends from the drive piston radially inwardly.

19. An infusion pump assembly comprising:
an insulin pump including a drive piston;
a syringe assembly including a substantially hollow syringe housing and a plunger axially movable within the syringe housing to expel a fluid therefrom, the plunger being configured to operatively engage the drive piston via relative axial movement, the plunger having a hollow body including a closed end having a given area, the hollow body configured to receive the drive piston with a gap between the drive body and the plunger hollow body; and
a sealing elastic member positional within the gap to seal the plunger relative to the drive piston.

20. The infusion pump assembly of claim 19 wherein the gap has an annular area which is approximately one-fifth or less than the given area.
